(19) United States
(12) Patent Application Publication
(10) Pub. No.: US 2013/0108706 A1
(43) Pub. Date: May 2, 2013

(54) DIETARY FORMULATIONS

(71) Applicant: Katja Svennevig, Heggedal (NO)

(72) Inventor: Katja Svennevig, Heggedal (NO)

(21) Appl. No.: 13/701,952

(22) PCT Filed: Jun. 6, 2011
(86) PCT No.: PCT/GB2011/000849

§ 371 Date: Jan. 6, 2013

(30) Foreign Application Priority Data
Jun. 4, 2010 (GB) 1009368.0

(51) Int. Cl. A61K 35/56; A61K 31/375; A61K 31/122; A61K 31/355; A61K 31/047

(52) U.S. Cl. CPC A61K 35/612; A61K 31/355; A61K 31/047; A61K 31/122; A61K 31/375; A61K 35/618
USPC 424/538; 424/547

(57) ABSTRACT

The present invention relates to the field of nutritional support for individuals and reducing the effects of muscle conditions caused by high levels of physical activity. In particular, the invention provides dietary formulations, compositions comprising these formulations and uses of these compositions to support the body's muscle regeneration process.

### FIELD OF THE INVENTION

The present invention relates to the field of nutritional support for individuals and reducing the effects of muscle conditions caused by high levels of physical activity. In particular, the invention provides dietary formulations, compositions comprising these formulations and uses of these compositions to support the body's muscle regeneration process.

### BACKGROUND OF THE INVENTION

Levels of obesity and other conditions resulting from a less active life style have dramatically increased in the general population in recent years. Concomitantly, during recent decades, individuals have made efforts to counter the reduced amount of physical activity in their normal daily lives by participating in increased amounts of exercise. In many cases, the intensity of this exercise has increased and it is not uncommon that regular exercising individuals have a similar performance profile compared to professional athletes.

Too much, or too intense, exercise, creates new challenges for the subject such as exercise-induced muscle damage and muscle stress. Delayed Onset Muscle Soreness (DOMS), also called muscle fever, is the pain and stiffness felt in muscles about 24 to 72 hours after unaccustomed and/or strenuous exercise. In other words, it is a symptom of muscle damage caused by excessive exercise.

Further injuries and conditions have been observed to accompany DOMS. Examples are ultrastructural disruptions of myofilaments, especially at the Z-disc, as well as damage to the muscle's connective tissue. Even though DOMS usually disappears by itself after about 72 hours, only a few measures are known to accelerate muscle recovery. Given the intensity and increased frequency of physical activity, there is a high demand for means of reducing the recovery period to a minimum.

Known examples of measures which accelerate muscle recovery are measures that increase blood flow to the muscle, such as low-intensity work, massage, hot baths or a sauna visits. Such measures usually reduce the soreness or damage and related conditions only slightly and so these measures are typically regarded only as support measures. These measures do not tackle the underlying problem and mechanism and so do not lead to a fast muscle recovery.

In addition to DOMS, other and more severe cases of exercise-induced muscle damage may appear. Many athletes experience the discomfort and debilitating effects of exercise-induced muscle damage, which is linked to increased inflammatory processes in the muscle. Exercise-induced muscle injury in humans frequently occurs after unaccustomed exercise, particularly if the exercise involves a large amount of eccentric (muscle lengthening) contractions

On top of the increase in inflammatory processes, free radicals are also produced during and following various forms of contractile activity. Free radicals are known to result in skeletal muscle damage.

it is not only exercise-related activities which lead to the conditions discussed herein. Any other increase in physical activity may lead to these conditions. By way of example only, daily hard physical work such as in construction or other body-challenging activities or injuries could be the cause. It is also possible for muscle stiffness and other life-style-related effects which have a negative impact on regular muscle fitness and function to be caused by non-physical activities.

In recent years, the effects of exercise and increased physical activity have been major focus areas within the nutraceutical, food supplements and functional food fields. Various publications have considered the effects of active ingredients such as antioxidants, phospholipids, vitamins and the like. Some of these are discussed below.

In Jäger et al., J. Int. Soc. Sports. Mgr. 2007:4:5, the beneficial effect of phospholipids on sports performance is described. This document shows that intense exercise reduces choline levels which is the precursor for acetylcholine, a known signaling molecule and neurotransmitter. The supplementation of choline has been shown to have benefits in improving performance.

While some publications point to a possible effect of antioxidants for the improvement of muscle recovery after exercise, Teixeira et al; Med Sci Sports Exerc. 2009 September; 41(9): 1572-60 showed that antioxidants do not prevent post-exercise peroxidation and may even delay or hinder muscle recovery. This study aimed to determine the effects of 4 weeks of antioxidant supplementation on exercise-induced lipid peroxidation, muscle damage, and inflammation in kayakers. The antioxidants used were alpha-tocopherol, vitamin C, beta-carotene and lutein, in addition to generic ingredients.

WO2008117062 suggests a composition comprising phospholipids and astaxanthin, all directly derived from the extraction of krill oil, for combating different conditions including diet-induced hyperinsulinemia, insulin insensitivity, muscle mass hypertrophy, serum adiponectin reduction and hepatic steatosis.

Further dietary supplement combinations of antioxidants, fish oils and other ingredients are shown in WO2007076416 with the purpose of providing such supplements for inhibiting the progression of macular degeneration and promoting healthy vision.

The present Applicant has previously sold a combination of the antioxidants lutein, astaxanthin and zeaxanthin under the trademark VitaePro™ as a food supplement for improving the immune system as well as for increasing training effects. Contrary to the findings of previous publications, markers of oxidative stress in the liver were reduced in studies using the product. Further, taking into consideration the results from epidemiologic studies in diabetics where the effects of carotenoids have been studied, it seems that the product VitaePro™ is efficacious in the prevention of type 2 diabetes.

Zoppi et al. J Int Soc Sports Nutr 2006; 3:37-44 discloses that the combination of vitamins E and C can reduce oxidation and muscle damages in professional football players. Rosa et al J Appl Physiol 2009; 107:1532-1538 discloses that Vitamin C and E supplementation also prevents mitochondrial damage of ileum myocytes caused by intense and exhaustive exercise training.

Naziroglu et al. Can J Appl Physiol 2005; 30:172-185 and Kutlu et al. Int J Vitam Nutr Res 2005; 75:71-80 disclose the protective effects of moderate exercise with dietary vitamin C and E on the blood antioxidative defense mechanism in rats with streptozotocin-induced diabetes and also that this combination counteracts oxidative stress in the kidney and lens of streptozotocin-induced diabetic-rat.

Kiokias et al. Eur J Clin Nutr 2003; 57:1135-1140 discloses that dietary supplementation with a natural carotenoid mixture decreases oxidative stress. In detail, this study showed that the consumption of the natural carotenoid mixture lowered the increase in oxidative stress induced by the fish oil as assessed by ex vivo oxidative stability of LDL and DNA degradation products in urine. The carotenoid mixture also enhanced the plasma triglyceride-lowering effects of the fish oil.

Atalay at al Med Sci Sports Exerc 2000; 32:601-607 shows that Vitamin E supplementation markedly decreased fish oil and exercise induced antioxidant enzyme activities in all tissues.

Sen et al J Appl Physiol 1997; 83:189-195 describes the administration of a combination of fish oil and vitamin E supplementation to subjects in a state of oxidative stress, at rest and after physical exercise. This publication discloses that fish oil induced oxidative damage of lipids in a tissue-specific manner while a combination of fish oil and vitamin E decreased tissue oxidative stress to below that seen in non-vitamin E-supplemented animals. The fish oil and vitamin E supplementation also appeared to have some effect on the increase in exercise-induced oxidative stress in the liver of test animals.

Despite the availability of the formulations, methods and agents described above, there remains a need for new compositions that are more effective in reducing the effects of exercise-induced conditions like oxidative stress, oxidative damages and the impairment of the immune system by increased levels of physical activity. In particular a need for a product which can ameliorate muscle damage or stress remains. The present inventors have designed an optimal nutraceutical supplement formula to address this issue.

### SUMMARY OF THE INVENTION

The main goal of the inventors of the present invention was to counteract the negative effects of DOMS and other muscle damages by providing a composition comprising several agents which reduce related symptoms and accelerate the muscle restoration process. In addition to addressing specific muscle-related conditions, the formulations of the present invention provide for a general improvement of the well-being of the subject and increase the longevity of these individuals by reducing negative side-effects related to over-exposed muscles. To achieve that goal, it was necessary to approach that challenge not only by addressing some of the underlying mechanisms, but also to consider the totality of factors leading to these conditions including, but not limited to, inflammatory processes, muscle damage and the development of free radicals. There is reason to believe that by addressing these various causative factors simultaneously and in the manner as described in the present invention, it is possible to achieve a significant and accelerated muscle regeneration process. The formulations of the present invention may also effect muscles other than the muscles directly suffering the consequences of the individual's physical activity. For instance the heart muscle may be strengthened by the formulations of the present invention.

The present invention describes for the first time a specific combination of ingredients in a dietary formulation, which has a significant impact on the accelerated reduction of muscle stress and damage after exercise/training and at the same time provides an immune-enhancing effect for the individual.

In a first aspect the present invention provides a dietary formulation comprising:

a) an antioxidant;
b) an agent for support of cellular unction and structure; and
c) an anti-inflammatory agent.

As used in this application, the terms “functional ingredient, “functional agent” or “functional component” as used interchangeably herein refer to substances known to have one of the following functional features or activities: (a) antioxidant activity; (b) activity as agents for support of cellular function and structure; and (c) anti-inflammatory activity.

Thus, an antioxidant is a compound having antioxidant activity, an agent for support of cellular function and structure is a compound with activity in support of cellular function and structure and an anti-inflammatory agent is a compound with anti-inflammatory activity.

The phrase “antioxidant” as used herein means an agent that can prevent or reduce oxidative (muscle) stress and damage in an individual. Antioxidants include, but are not limited to, natural antioxidants including ascorbic acid or derivatives thereof such as ascorbyl palmitate, as well as tocopherols, herbal extracts, Resveratrol, carotenoids and synthetic antioxidants such as butylated hydroxytoluene, or mixtures thereof. A wide variety of antioxidant agents are commercially available from sources known by those of ordinary skill in the art.

The antioxidant used in context of the present invention can be a water-soluble substance, optionally selected from the group consisting of Vitamin C, polyphenols, proanthocyanidins, anthocyanins, bioflavonoids, a source of selenium (e.g., sodium selenite, sodium selenate or L-selenomethionine), alpha-lipoic acid, glutathione, catechin, epicatechin, epigallocatechin, epigallocatechin gallate, epicatechin gallate and cysteine.

Alternatively, the antioxidant can be a fat-soluble substance, optionally selected from the group consisting of Vitamin E, gamma tocopherol, alpha-carotene, beta-carotene, lutein, zeaxanthin, retinal, astaxanthin, cryptoxanthin, natural mixed carotenoids, lycopene and resveratrol.

The formulation may contain multiple antioxidants. Optionally, the formulation contains both fat-soluble and water-soluble antioxidants. Optionally, the formulation contains as antioxidants Vitamin E, Vitamin C, and natural carotenoids such as astaxanthin, zeaxanthin and lutein.

The antioxidants used in the present invention can be hydrophobic or hydrophilic. Preferably, the formulations contain a mixture of hydrophobic and hydrophilic antioxidants. The antioxidants serve, together with other components in the formulation, to inhibit the results of oxidative (muscle) stress and damage due to exercise/training or other types of physical activity.

Carotenoids are a preferred group of antioxidants used in the present invention. These antioxidants are a class of compounds, which are classified into two main groups: carotenes and xanthophylls. In contrast to carotenes, which are pure polyene hydrocarbons, such as beta-carotene or lycopene, xanthophylls contain oxygen functional groups, such as hydroxyls, epoxy and/or oxo groups. Typical, non-limiting representatives of the xanthophyll group are astaxanthin, canthaxanthin, lutein, fucoxanthin and zeaxanthin. Natural sources of dietary astaxanthin, include krill, crawfish, crustacean processing by-products, bacteria, yeast, algae, and higher plants. Carotenoids may also be synthetically derived and may contain different isomers of carotenoids than those contained in the natural preparations. Depending on the intended use, natural, synthetic or mixtures of natural and synthetic carotenoids may be included in a variety of ways in the present invention. Optionally they may be included as oils, encapsulated oils or blends.

The concentrations of carotenoids in the formulations will vary, but will be in amounts useful in dietary supplements.

The agent for support of cellular function and structure is preferably an oil obtained from a marine organism. The oil obtained from a marine organism may be obtained from fish, and preferably the fish is a cold-water fish, more preferably salmon, tuna, halibut or herring. Preferably, however, the oil is obtained from marine invertebrates such as molluscs or crustacea. Preferably, the mollusc is calamari (squid). Most preferably the crustacea is krill.

Optionally the agent for support of cellular function is a phospholipid such as, without limitation, phosphatidylcholines such as phosphatidyl choline (PC), dipalmitoylphosphatidylcholine (DPPC), and other disaturated phosphatidyl cholines, phosphatidyl ethanolamines, phosphatidylinositol, phosphatidyl serines such as sphingomyelin or other ceramides, various other phospholipids, phospholipid-containing oils such as lecithin oils derived from soy beans, and mixtures and combinations thereof.

The phospholipids of the present formulation can be found in Poly Unsaturated Fatty Acid (PUFA)-rich extracts of single cell organisms such as, but not limited to Crypthecodinium sp., Schizochytrium sp., Mortierella sp. and Parecoccus sp. Phospholipids of the present invention can also be derived from animal sources including, but not limited to, animal organ extracts (e.g., brain, liver, other animal process wastes), egg yolk, egg yolk extracts, fish byproducts and fish byproduct extracts (i.e., processed waste products from preparation of fish meal or purified fish oil).

In a preferred embodiment, the agent for support of cellular function and structure is a phospholipid extracted from or present in the oil of a marine organism as described above. Phospholipids from marine invertebrates are preferred. Particularly preferred phospholipids are from molluscs, more preferably calamari and from crustacea, more preferably krill. The most preferred phospholipids are from Euphausiids from the order Euphausiacia, commonly known as krill. Phospholipids useful for this invention include those from krill oil including such oils which contain approximately 70% (w/w) omega-3 phospholipids of total phospholipids; and having an amount of phospholipids compared to total lipid amount of approximately 40% (40 g/100 g). The total amount of omega-3 fatty acids are 36% (w/w) of total fatty acids.

The formulation may contain multiple agents for support of cellular function and structure. Further possible agents for support of cellular function and structure include those derived from other sources of phospholipids, for instance from egg yolks, beef liver, cow brain and soy; as well as from any other source of triglyceride omega-3-fatty acids, for instance, from flaxseeds, walnuts, hemp seeds, soybeans and some dark green leafy vegetables, corn oil, safflower oil, sunflower oil, and canola oil or most notably from cold water fish including salmon, tuna, halibut, and herring.

The anti-inflammatory agent is preferably oil obtained from a marine organism. The oil may be obtained from fish, and preferably the fish is a cold-water fish, more preferably salmon, tuna, halibut or herring. Preferably, however, the oil is obtained from marine invertebrates such as molluscs or crustacea. Preferably, the mollusc is calamari (squid). Most preferably the crustacea is krill.

The anti-inflammatory agent optionally comprises an omega-3 fatty acid. Preferably the omega-3 fatty acid is selected from the group consisting of α-linolenic acid, eicosapentaenoic acid, docosapentaenoic acid and docosahexaenoic acid.

Omega-3 fatty acids are well-known compounds which can be obtained from different sources, but are usually found naturally in abundance in the tissue of cold water fish and other marine organisms, such as crustaceae (e.g. krill) and molluscs (such as calamari).

Optionally, the source of the omega-3 fatty acid is flax seed, flax oil, walnuts, canola oil, wheat germ, or oil from fish, molluscs or crustacea. The formulation may contain multiple anti-inflammatory agents.

Preferred omega-3 fatty acids used in the present formulations to increase the pace of regenerating muscle function according to the present invention are DHA (docosahexaenoic acid), EPA (eicosapentaenoic acid) and alpha-linoleic acid (ALA). For simplicity, the term “DHA” is used herein to refer to any of these three omega-3 fatty acids or to a mixture of two or three of these omega-3 fatty acids. In other words, when the term “DHA” is used herein, the skilled artisan would understand that either DHA, EPA, ALA or a mixture of two or three of EPA, DHA and ALA could be used in that instance. Preferably, at least one third of the omega 3 mixture consists of DHA and/or EPA. When a mixture of EPA to DHA is used, the ratio of EPA to DHA can be from about 1.0 to about 10.0, preferably from about 1.0 to about 5.0, more preferably from about 1.0 to 4.0. Still more preferably, the ratio of EPA to DHA when a mixture is used is in the range 1.0 to 2.0 EPA:DHA.

One preferred blend according to the present invention is harvested from calamari and typically has a ratio of EPA to DHA of 1:4. The preferred blend according to the present invention is usually harvested from Crustacea and typically has a ratio of EPA to DHA of between 1.7 and 1.9. Preferably the blend is harvested from krill, because omega-3 phospholipids, as the main omega-3 fatty acid source in krill oil, follow simpler digestion and distribution routes than omega-3 triglycerides in the human body. Greater bioavailability and bioefficiency of omega-3 phospholipids relative to omega-3 triglycerides influence cellular absorption, functioning and distribution of omega-3.

Alternatively or in addition, any other anti-inflammatory agent with natural origin may be used. By way of example, such anti-inflammatory agents include oleocanthal, beta-glucans, extracts from nuts, seeds, and certain spices such as ginger, astaxanthin, lutein, zeaxanthin, vitamins such as vitamin C or D, as well as enhancers of prostaglandins having anti-inflammatory properties.

Optionally, the oil obtained from a marine organism fulfils two functions. First, the oil fulfils the function of an anti-inflammatory agent and secondly it fulfills the function of an agent for the support of cellular function and structure.

In a preferred embodiment of the present invention, krill oil fulfils two functions. First, the krill oil fulfils the function of the anti-inflammatory agent and secondly it fulfills the function of the agent for support of cellular function and structure. In another preferred embodiment of the present invention, calamari oil fulfils two functions. First, the calamari oil fulfils the function of the anti-inflammatory agent and secondly it fulfills the function of the agent for support of cellular function and structure.

Phospholipids from krill oil or from calamari oil can, besides their effects as agents for support of cellular function and structure, be used as anti-inflammatory agents in the formulations of the present invention. Omega-3 fatty acids from krill oil or from calamari oil can, besides their anti-inflammatory effect, also be used as agents for support of cellular function and structure in the formulations of the present invention.

Therefore, specifically included within the present invention is the scenario wherein the formulation comprises each of the components a), b) and c) above, but one product or compound, is both component a) and component b), or both component b) and component c), or both component a) and component c). Alternatively viewed, included within the scope of the invention are formulations in which two of the components a) to c) are from the same source or are the same product or compound. Preferably krill oil or calamari oil is both the anti-inflammatory agent and the agent for support of cellular function and structure.

In a further aspect, the present invention provides a dietary formulation comprising:

a) an antioxidant; and
b) oil derived from a marine organism.

The antioxidant and oil derived from a marine organism may be as described anywhere herein.

The formulation may comprise multiple antioxidants and/or multiple oils derived from marine organisms, or components thereof, as described anywhere herein. The formulation may comprise further anti-inflammatory agents and/or agents for the support of cellular structure and function, as described anywhere herein.

In a further aspect, the present invention provides a composition, which is an animal feed product, a dietary supplement, or a human food product, comprising the formulation recited above or anywhere herein.

In a further aspect the present invention provides a formulation as recited above or anywhere herein for use in combating an excessive exercise-induced or physical activity-induced condition in a subject, or for reducing oxidative muscle stress or muscle damage.

Alternatively viewed, the present invention provides a method of combating an excessive exercise-induced or physical activity-induced condition or reducing oxidative muscle stress or muscle damage in a subject comprising the consumption of a formulation as recited above or anywhere herein.

The formulations recited above or anywhere herein are used in an amount effective to combat the negative results of an exercise-induced or physical activity-induced condition such as oxidative stress and oxidative damage. Optionally the excessive exercise-induced or physical activity-induced condition is selected from the group consisting of Delayed Onset Muscle Soreness (DOMS), muscle cramps or spasms, muscle aches, muscle atrophy, muscle injuries like strains and the disruption of muscle fibers, muscle weakness and heaviness, metabolic muscle disorders, muscle soreness and muscle fatigue.

Also provided is the formulation as defined anywhere herein for use in accelerating the reduction of muscle stress and damage after exercise and/or training. Preferably the formulations of the present invention also provide at the same time an immune-enhancing effect for the individual to whom the formulations are administered.

In certain embodiments, the formulation is administered as part of a dietary regimen, for instance, one or more times per day, one or more times per week, or less frequently, depending on the amount of exercise or physical activity. Administration may be for any length of time deemed effective, for example one week, one month, three months or a year or more, extending to the duration of the subject's life.

Other features and advantages of the invention will be understood by reference to the detailed description and examples that follow.

### DETAILED DESCRIPTION OF THE INVENTION

The challenges of muscle stress and damage after exercise/training cannot be solved by the administration of one of the agents described above alone as the conditions do not originate from only one source. Rather, a combination of different causes leads to these conditions. Therefore, a combination of agents, preferably in specific concentrations, enable the individual to recover at a much faster pace compared to regular nutrition, intake of one agent alone or even by applying active known counter-measures. Further, the formulations of the present invention permit positive synergies between the components, said synergies being beneficial for treating the above-mentioned conditions.

In particular, the Examples herein show the exceptional and surprising efficacy of the formulations of the invention, in particular the impact of the marine oil and of combinations of antioxidants.

In addition to the use of these formulations as means for counter-acting an existing condition, the compositions of the present invention are particularly beneficial as prophylaxis before exercise or physical activities.

“Effective amount” as used herein refers to an amount of a compound, material, or composition as described herein that is effective to achieve a particular biological result in relation to the purpose of the present invention. Such effective activity may be achieved, for example, by administering the compositions of the present invention to an individual.

A “subject” or “individual” as used herein refers to an animal of any species. In various embodiments, the animal is a mammal, and may be, and is preferably, a human.

The terms “muscle damage”, “muscle disorders”, “exercise-induced muscle disorders”, “muscle conditions”, “muscle stress” and similar terms are used herein to refer to conditions of muscle alterations, generally being the result of excessive exercise of physical activity. Such conditions include but are not limited to muscle cramps or spasms, muscle aches, muscle atrophy, muscle injuries such as strains and the disruption of muscle fibers, muscle weakness and heaviness, metabolic muscle disorders, muscle soreness, muscle fatigue, muscle stiffness and lack of muscle flexibility.

As used herein, a “dietary supplement” is a product that is intended to be ingested in addition to the normal diet of an animal. The animal is a mammal, and may be, and is preferably, a human.

In context of the present invention, the ingredients may be combined in various formulations, By way of example only, one formulation comprises at least one antioxidant, preferably one water-soluble antioxidant and one fat-soluble antioxidant. Another formulation comprises at least one functional ingredient that reduces inflammation.

The following parts of the description describe oral administration of tablets, caplets or capsules comprising the formulations of present invention. This mode of administration and these physical products are only examples and the use of such examples shall not exclude other modes of administration or types of products.

In one embodiment, the average total filling weight of a composition (tablet, caplet, capsule etc) comprising the formulation is about 555 mg. If a coating is present on the composition then preferably the filling weight of the capsule, which comprises the formulation and any excipients, is about 555 mg and the capsule shell weighs about 205 mg. Thus the composition weighs about 760 mg. These weights are merely exemplary and the skilled man would be aware of other weights of compositions, fillings and shells which could be used.

All w/w % values below are based on this preferred average filling weight. The skilled man in the field of nutraceuticals and supplementation will appreciate that different types of tablets or caplets may be used to deliver the formulation according to the present invention and that these may have different weights and may comprise different amounts of the formulation. A person skilled in the art will therefore appreciate that the ratios of the ingredients present in a delivery vehicle comprising the formulation of the invention may vary depending on the weight of the delivery vehicle, the amount of the formulation present, the type of administration, and several other parameters. The skilled man would also bear in mind the maximum and recommended daily intake dosages for each of the components of the formulation when determining the precise amounts to include in the tablet, capsule or other delivery vehicle. The term “capsule” when used below is only exemplary and any other delivery vehicle or composition could be used instead.

In another embodiment, the average total filling weight of a capsule comprising the formulation is about 560 mg. A coating (shell) is present on the capsule and weighs about 205 mg. In total the composition weighs about 765 mg. In another embodiment, the average total filling weight of a capsule comprising the formulation is about 673 mg. A coating (shell) is present on the capsule and weighs about 250 mg. In total the composition weighs about 923 mg. Again, these weights are merely exemplary and the skilled man would be aware of other weights of compositions, fillings and shells which could be used. The filling, shell and total capsule weights can vary by +/− about 20%, more preferably +/− about 10%.

The “oil derived from a marine organism” (component (b)) is typically present in the formulation at concentration of about 30 and 90%, more preferably about 40 to 90%, still more preferably about 40 to 60% or about 54 to 90%, most preferably about 55% (w/w) or in an amount of about 150 to 500 mg, more preferably about 225 to 500 mg, more preferably about 225 to 350 mg or about 300 to 500 mg, most preferably about 300 mg per unit dosage form.

If present, preferably, the combined concentration (w/w) of carotenoids in the capsule is in the range of about 0.1 to 10%, more preferably about 0.1 to 9%, more preferably about 0.1 to 5%, more preferably 0.18 to 4.5%. Alternatively viewed, if present, preferably the amount of carotenoids in the capsule as a whole is about 1 to 50 mg, more preferably about 1 to 25 mg, most preferably about 10 mg.

Different carotenoids may be present in different concentrations or amounts. For instance, if present, astaxanthin is preferably present in a concentration of about 0.1 to 2%, more preferably about 0.18 to 1.8%, most preferably about 0.25 to 0.4% (w/w) or in an amount of about 1 to 25 mg, more preferably about 1 to 20 mg, still more preferably about 1 to 5 mg, most preferably about 2 mg per capsule. The astaxanthin may be present in the composition in the form of an astaxanthin-containing product, which does not consist solely of astaxanthin and the amount of that product to be included in the composition in order to provide the required level of astaxanthin will be readily determinable by the skilled man. For instance, astaxanthin-containing compositions comprising about 10% astaxanthin are available. If the astaxanthin is present in the form of a product containing about 10% astaxanthin then the composition preferably comprises about 10 to 250 mg, more preferably about 10 to 200 mg, still more preferably about 10 to 50 mg, most preferably about 20 mg per capsule of that product.

If present, lutein/zeaxanthin (i.e. lutein and zeaxanthin together) are preferably present in a concentration of about 0.1 to 3%, more preferably about 0.18 to 2.7%, still more preferably about 1.0 to 1.4% (w/w) or in an amount of about 1 to 35 mg, more preferably about 1 to 10 mg, most preferably about 6.5 to 7.5 mg per capsule.

The individual concentrations of the different carotenoids will not necessarily be the same. Preferably, the ratio of lutein to zeaxanthin present is about 7:1 to 4:1, more preferably about 5:1. Preferably, lutein is present at a concentration of about 0.15 to 1.8%, more preferably about 0.75 to 1.35%, most preferably about 0.9 to 1.07% (w/w) or in an amount of about 1 to 10 mg, more preferably about 5 to 7.5 mg, most preferably about 6 mg per capsule. Preferably, zeaxanthin is present at a concentration of about 0.03 to 0.4%, more preferably about 0.15 to 0.28%, most preferably about 0.18 to 0.2% (w/w) or in an amount of about 0.2 to 5 mg, more preferably about 1 to 1.5 mg, most preferably about 1.2 mg per capsule.

The lutein and zeaxanthin may be provided to the composition separately, or together in a single commercial product. The lutein and zeaxanthin may be present in the composition in the form of products which do not consist solely of lutein and zeaxanthin and the amount of that product to be included in the composition in order to provide the required level of lutein and zeaxanthin will be readily determinable by the skilled man.

As mentioned above, in the formulations of the present invention, other antioxidant ay be added, such as Vitamin E and/or Vitamin C, which exhibit antioxidant function.

There is no known optimal daily dose of Vitamin C, although the U.S. Recommended Dietary Allowance is 60 mg. However, dosages of 1.0 grams and more have frequently been taken as a supplement for general health without negative side-effects and all such dosages are useful in the formulations of the present invention. Although ascorbic acid or rose hips can be used, the present composition preferably utilizes Vitamin C in the form of sodium ascorbate because it is easily dissolved in the digestive system and causes relatively minimal irritation. If Vitamin C is present, it is optionally present in a concentration of about 2 to 90%, about 2 to 50%, about 2%, or about 2.16% (w/w) or optionally in an amount of about 10 to 1000 mg, 12 to 500 mg, or 12 mg per capsule.

Preferably, Vitamin C is present at a concentration of about 1 to 50%, more preferably about 2 to 25%, most preferably about 5 to 12% (w/w) or in an amount of about 5 to 300 mg, more preferably about 10 to 150 mg, most preferably about 30 to 80 mg per capsule. The Vitamin C may be present in the composition in the form of product which does not consist solely of Vitamin C and the amount of that product to be included in the composition in order to provide the required level of Vitamin C will be readily determinable by the skilled man.

Preferably, the Vitamin C/Ascorbic acid is provided in the form of a product, preferably a fruit extract, e.g. an Acerola or Emblica officinalis extract, which comprises about 17% or about 50% Vitamin C. If such products are used, then the composition preferably comprises about 30 to 1000 mg, more preferably about 60 to 500 mg, most preferably about 175 mg of said product per capsule.

The present U.S. Recommended Dietary Allowance of Vitamin E is 10 mg/day. If Vitamin E is present, it is preferably present in a concentration of about 0.2 to 25%, more preferably about 0.32 to 22.5%, still more preferably about 1 to 5%, most preferably about 1.8 to 2.15% (w/w) or in an amount of about 1.5 to 250 mg, more preferably about 1.8 to 125 mg, still more preferably about 5 to 30 mg, most preferably about 12 mg per capsule. The Vitamin E may be present in the composition in the form of a product which does not consist solely of Vitamin E and the amount of that product to be included in the composition in order to provide the required level of Vitamin E will be readily determinable by the skilled man. Preferably the Vitamin E is present in the form of a D-α-Tocopherol, which are widely available and known to have vitamin E activity.

Further ingredients which may be present in the formulation according to the present invention are vitamins, such as, vitamin A, vitamin A acetate, vitamin A palmitate, riboflavin, vitamin B, ascorbic acid, ascorbyl palmitate, nicotinic acid, nicotinamide, pyridoxine hydrochloride, vitamin D3 (also known as cholecalciferol and calciol), tocopherol, tocopherol acetate, tocopherol palmitate, tocotrienol, vitamin K, thiamine, calcium pantothenate, biotin, lipoic acid, folio acid, and folio acid derivatives as well as compounds with vitamin or coenzyme characteristics, such as choline chloride, carnitine, taurine, creatine, ubiquinones, S-methylmethionine, and S-adenosylmethionine. These vitamins can, where applicable, either be used as antioxidants according to the present invention or as further factors supporting one or more ingredients of the formulation according the present invention or simply act as further beneficial ingredients.

If Vitamin D₃ is present, it is preferably present in a concentration of about 0.0005 to 0.0015%, more preferably about 0.0006 to 0.0010%, most preferably about 0.0007 to 0.0009% (w/w) or in an amount of about 2 to 20 μg, more preferably about 3 to 10 μg, most preferably about 5 μg per capsule. The Vitamin O₃ may be present in the composition in the form of a product which does not consist solely of Vitamin D₃ and the amount of that product to be included in the composition in order to provide the required level of Vitamin D₃ will be readily determinable by the skilled man.

If present, preferably krill oil is present at a concentration of about 30 and 90%, more preferably about 40 to 90%, still more preferably about 40 to 60% or about 54 to 90%, most preferably about 55% (w/w) or in an amount of about 150 to 500 mg, more preferably about 225 to 500 mg, more preferably about 225 to 350 mg or about 300 to 500 mg, most preferably about 300 mg per capsule.

If present, preferably calamari oil is present at a concentration of about 25 to 75%, more preferably about 35 to 50%, most preferably about 45% (w/w) or in an amount of about 150 to 500 mg, more preferably about 225 to 350 mg, most preferably about 300 mg per capsule.

Optionally the formulation of the invention also comprises physiological acceptable excipients such as but not limited to lecithin, monoglycerides, glycerol monostearate and soybean oil. If the capsule filling is about 555 mg or about 560 mg, preferably the total amount of excipients in the filling is about 5 to 15 mg, more preferably about 8 mg, however, this is may be varied greatly between different compositions and the skilled man would be aware of the appropriate amounts of excipients which can be present.

A preferred formulation comprises krill oil, Vitamin C, and one or more of (e.g. all of) astaxanthin, lutein and zeaxanthin. More preferably, the formulation also comprises Vitamin D and/or Vitamin E. Optionally, the formulation also comprises lecithin as an excipient. All possible combinations of the concentrations and amounts disclosed herein of each of these components are explicitly contemplated as part of the invention. Also contemplated are formulations in which the amount or concentration of any given one, two, three, four, five, six or seven components are specified whereas the amounts or concentrations of the other components are not specified. All potential arrangements of components with specified/non-specified amounts or concentrations are explicitly contemplated as part of the invention.

An alternative formulation comprises calamari oil, Vitamin C (above), and one or more of (e.g. all of) astaxanthin, lutein and zeaxanthin. More preferably, the formulation also comprises Vitamin D and/or Vitamin E. Optionally, the formulation also comprises any or all of lecithin, monoglycerides, glycerol monostearate and soybean oil as excipient(s). All possible combinations of the above-mentioned concentrations and amounts of each of these components are explicitly contemplated as part of the invention. Also contemplated are formulations in which the amount or concentration of any given one, two, three, four, five, six or seven components are specified whereas the amounts or concentrations of the other components are not specified. All potential arrangements of components with specified/non-specified amounts or concentrations are explicitly contemplated as part of the invention.

In an exemplary embodiment the present invention provides a formulation comprising krill oil, 300-500 mg/capsule or 54 to 90% (w/w); vitamin E, 1.8-125 mg/capsule or 0.32 to 22.5% (w/w); vitamin C, 12 to 500 mg/capsule or 2.16 to 90% (w/w); lutein/zeaxanthin 5:1, 1 to 30 mg/capsule or 0.18 to 2.7% (w/w); astaxanthin, 1 to 20 mg/capsule or 0.18 to 1.8% (w/w). Optionally this formulation also comprises physiological acceptable excipients such as but not limited to lecithin.

In a further exemplary embodiment, the present invention provides a formulation comprising krill oil (about 40 to 60% (w/w) or about 225 to 350 mg per capsule), astaxanthin (about 0.25 to 0.4% (w/w) or about 1 to 5 mg per capsule), lutein (about 0.75 to 1.35% (w/w) or about 5 to 7.5 mg per capsule), zeaxanthin (about 0.15 to 0.28% (w/w) or about 1 to 1.5 mg per capsule), Vitamin C (about 2 to 25% (w/w) or about 30 to 80 mg per capsule), Vitamin E (about 1 to 5% (w/w) or about 5 to 30 mg per capsule), and optionally also Vitamin D (about 0.0006 to 0.0010% (w/w) or about 3 to 10 μg per capsule). Optionally this formulation also comprises physiological acceptable excipients such as but not limited to lecithin.

In a further exemplary embodiment, the present invention provides a formulation comprising calamari oil (about 35 to 50% (w/w) or about 225 to 350 mg per capsule), astaxanthin (about 0.25 to 0.4% (w/w) or about 1 to 5 mg per capsule), lutein (about 0.75 to 1.35% (w/w) or about 5 to 7.5 mg per capsule), zeaxanthin (about 0.15 to 0.28% (w/w) or about 1 to 1.5 mg per capsule), Vitamin C (about 2 to 25% (w/w) or about 30 to 80 mg per capsule), Vitamin E (about 1 to 5% (w/w) or about 5 to 30 mg per capsule), and optionally also Vitamin D (about 0.0006 to 0.0010% (w/w) or about 3 to 10 μg per capsule). Optionally this formulation also comprises physiological acceptable excipients such as but not limited to lecithin, monoglycerides, glycerol monostearate and soybean oil.

Optionally the formulation comprises 300 mg of Krill oil, 20.6 mg of Vitamin E, 176.5 mg of Vitamin C and 34.3 mg of lutein/zeaxanthin in a ratio of 5:1 and 22 mg of astaxanthin. Optionally this formulation also comprises physiological acceptable excipients such as but not limited to lecithin.

Formulations with the following specific compositions are provided:

### Krill Oil-Containing Formulation

| | | | Conc. | |
|---|---|---|---|---|
| | | | (% w/w) of | |
| | | Conc. | component | Amount of |
| | | (% w/w) of | in | component- |
| | Amount of | component | component- | containing |
| | component | in | containing | product |
| Component | per capsule | formulation | Product | per capsule |
| Krill oil | 300 mg | 53.6 | 100 | 300 mg |
| Vitamin C | 30 mg | 5.36 | 17 | 176.5 mg |
| Astaxanthin | 2 mg | 0.36 | 10 | 20 mg |
| Lutein | 6 mg | 1.07 | 21% | 34.3 mg |
| Zeaxanthin | 1.2 mg | 0.21 | (combined | |
| | | | total:Lutein | |
| | | | and | |
| | | | Zeaxanthin | |
| | | | provided | |
| | | | in a | |
| | | | combined | |
| | | | product in | |
| | | | a 5:1 ratio | |
| Vitamin D | 5 μg | 0.0009 | 1.88 | 0.266 mg |
| Vitamin E | 12 mg | 2.14 | 58.4 | 20.56 mg |
| Lecithin | 8.372 mg | 1.5 | 100 | 8.37 mg |
| | | | Total Filling | 560 mg |
| | | | Weight | |

### Calamari Oil-Containing Formulation

| | | | Conc. | |
|---|---|---|---|---|
| | | Conc. | (% w/w) of | |
| | | (% w/w) | component | Amount of |
| | | of com- | in | component- |
| | Amount | ponent | component- | containing |
| | of component | in formu- | containing | product |
| Component | per capsule | lation | Product | per capsule |
| Calamari oil | 312.5 mg | 46.4 | 100 | 312.5 mg |
| Vitamin C | 80 mg | 11.89 | 50 | 176 mg |
| Astaxanthin | 2 mg | 0.30 | 10 | 21 mg |
| Lutein | 6 mg | 0.89 | 21% | 34.3 mg |
| Zeaxanthin | 1.2 mg | 0.18 | (combined | |
| | | | total:Lutein | |
| | | | and | |
| | | | Zeaxanthin | |
| | | | provided | |
| | | | in a | |
| | | | combined | |
| | | | product in | |
| | | | a 5:1 ratio | |
| Vitamin D | 5 μg | 0.0007 | 1.88 | 0.266 mg |
| Vitamin E | 12 mg | 1.78 | 58.4 | 20.56 mg |
| Excipients | 108.37 mg | 16.1 | 100 | 108.37 mg |
| | | | Total Filling | 673 mg |
| | | | Weight | |

Optionally the formulation of the invention is in the form of a capsule. Preferably the components of the capsule shell are selected from the group consisting of gelatin, glycerine and water. If present, preferably the capsule shell comprises about 60%, more preferably about 63.41% or about 60.8% gelatin, about 30%, more preferably about 29.09% or about 27.89% glycerine and about 8% to 12%, more preferably about 8%, still more preferably about 7.49% or about 11.49% water.

The ingredients used in accordance with the present invention may be pre-blended with the other components of the composition to provide the beneficial amounts needed, may be coated onto a pet food composition, or may be added to the composition prior to offering it to the subject.

The dietary formulations and compositions of the invention can optionally comprise supplementary substances such as other vitamins, minerals, salts, condiments, colorants, and preservatives. Non-limiting examples of supplementary minerals include calcium, phosphorous, potassium, sodium, iron, chloride, boron, copper, zinc, manganese, iodine, selenium and the like. Non-limiting examples of supplementary vitamins include, various B vitamins, vitamin D, and vitamin K. Additional dietary supplements may also be included, e.g., niacin, pantothenic acid, insulin, folic acid, biotin, amino acids, and the like.

The formulations may also comprise one or more solidifying, bulking and agglomerating agents (collectively referred to herein as “solidifying agent(s)”). The solidifying agent(s) are used both in tableting and in generating solid-like carriers, such as beadlets, which are capable of transforming oils into stable agglomerates suitable for granulation, blending, and compression required for tableting. Examples of solidifying agents useful in the preparation of the formulations include, but are not limited to, sucrose, glucose, fructose, starches (e.g., corn starch), syrups (e.g., corn syrup), and ionic and nonionic polymers including, but not limited to, PEGs and other poly ether-like alkoxy cellulosics (HPMC), gellan, carrageenans, guar, hyaluronates, alginates, chondroitin sulfate, pectins, and proteins, (e.g., collagen or their hydrolyzed products (e.g., gelatins or polypeptides)). Other solidifying agents known to those skilled in the art of dietary supplement preparation may also be used in the preparation of the formulations of the present invention. The amount of solidifying agent(s) will vary, depending on the other components contained in the formulation, but will generally comprise the majority weight and volume of the dietary supplement.

The formulations of the present invention may also comprise additional excipients useful in preparing and finishing the dietary supplements. Such excipients may include timed-release polymer coating agents useful in prolonging dissolution of the formulation in the digestive tract. Examples of such polymers include, but are not limited to ionic and nonionic polymers, such as PEGs and other poly ether-like alkoxy cellulosics (HPMC), gellan, carrageenans, Eucheuma gelatenae, starch, hyaluronates, chondroitin sulfate, pectins, and proteins, e.g., collagen. Since the xanthophyll/carotenes are highly pigmented, coating technology may be applied to the dietary supplement in order to provide a dietary supplement of uniform color. Examples of color coating agents may include, but are not limited to, polymers, colorants, sealants and surface active agents including, not limited to, fatty acids and esters, di- and triglycerides, phospholipids including mono- and di-alkyl glyceryl phosphates, nonionic agents (like sugars, polysaccharides, e.g., HPMC and polysorbate 80) and ionic agents.

The dietary supplements may be manufactured using a number of techniques known in the art. The ingredients described herein are preferably present in the dietary supplements of the invention in an amount sufficient to provide the daily dosage (amount consumed per day) when the recommended number of dietary supplements is ingested per day. It is preferable, however, that the dietary supplement as described herein contains the described amounts of at least Vitamin C, Vitamin E, krill oil (phospholipids) lutein, zeaxanthin, and astaxanthin.

The skilled artisan will understand how to determine the appropriate amount of ingredients to be added to a given dietary formulation or composition for the purpose according to the present invention. Such factors that may be taken into account include the type of composition (e.g., pet food composition versus dietary supplement for humans), the average consumption of specific types of compositions by different individual, and the manufacturing conditions under which the composition is prepared. Preferably, the concentrations of a given ingredient to be added to the composition are calculated on the basis of the energy and nutrient requirements of the animal. According to certain aspects of the invention, the ingredients can be added at any time during the manufacture and/or processing of the composition. This includes, without limitation, incorporation within the formulation of the dietary supplement, or as a coating applied to the dietary supplement.

The present invention is directed to improved dietary supplement formulations for combating the effects of increased physical activity on muscles. As used herein, “dietary supplement(s)” or the shortened form, “supplement(s),” or the expression “nutraceutical(s) refer to any finished, dietary supplement or nutraceutical dosage form containing dietary substances and suitable for ingestion by a host, e.g., human or other mammal. Thus, the terms “dietary supplement” or “nutraceutical” is meant to encompass any form of dietary supplement, such as a tablet, chewable tablet, caplet, gelcap, powder, softgel, liquids etc. In some dosage forms, such as softgels, the use of concentrated oil phases of nutrients is desirable. The composition may also be of the timed-release or delayed-release types.

The compositions of the invention can be administered to the subject by any of a variety of alternative routes of administration, such as, without limitation, oral, intranasal, intravenous, intramuscular, intragastric, transpyloric, subcutaneous, rectal administration. Preferably, the dietary formulations or compositions are administered orally. As used herein, the term “oral administration” or “orally administering” means that the subject ingests, or a human is directed to feed, or does feed, an animal a formulation or composition described herein.

Administration can be on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the animal or otherwise contacted with or admixed with daily feed or food. When utilized as a daily feed or food, administration will be well known to those of ordinary skill.

Various compositions, tablets and capsules are described above as unitary dosage forms. 1 to 3 of these may conveniently be consumed per day and appropriate daily dosages can be calculated therefrom.

The following examples are provided to describe the invention in greater detail. They are intended to illustrate, not to limit, the invention. These Examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Various publications, including published applications and scientific articles, are cited throughout the specification. Each of these publications is incorporated by reference herein, in its entirety.

The present invention is not limited to the embodiments described and exemplified above, but is capable of variation and modification within the scope of the appended claims.

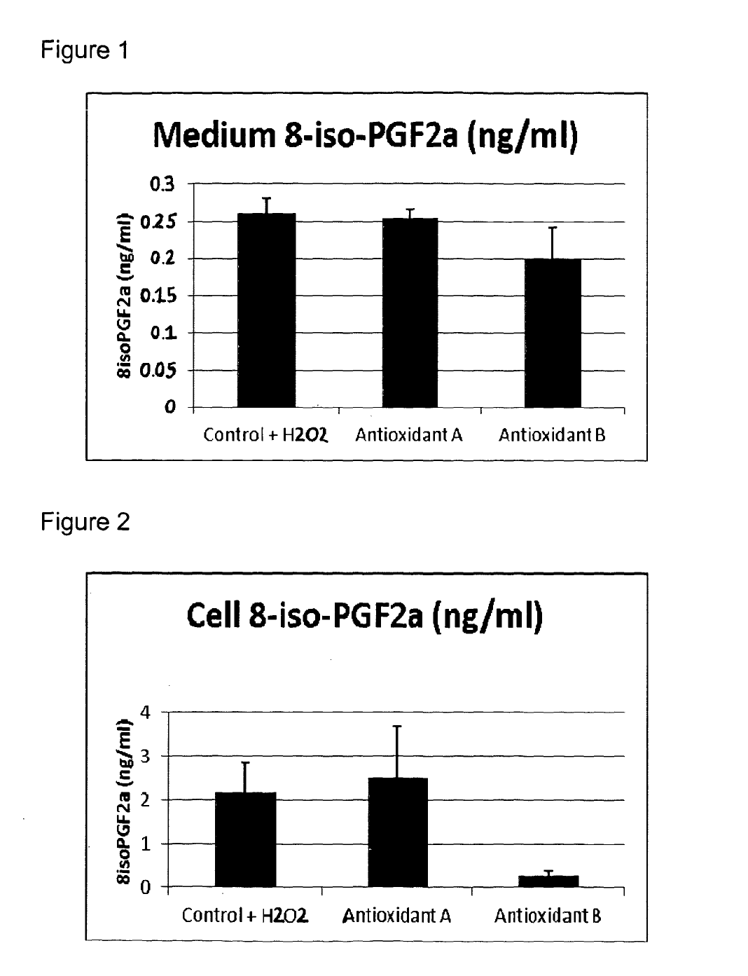

FIG. 1 shows the level of lipid peroxidation in the medium fraction measured by the marker 8-isoPGF2a. The values are the average from two separate cell isolations.

FIG. 2 shows the level of lipid peroxidation in the cell fraction measured by the marker 8-isoPGF2a. The values are the average from two separate cell isolations.

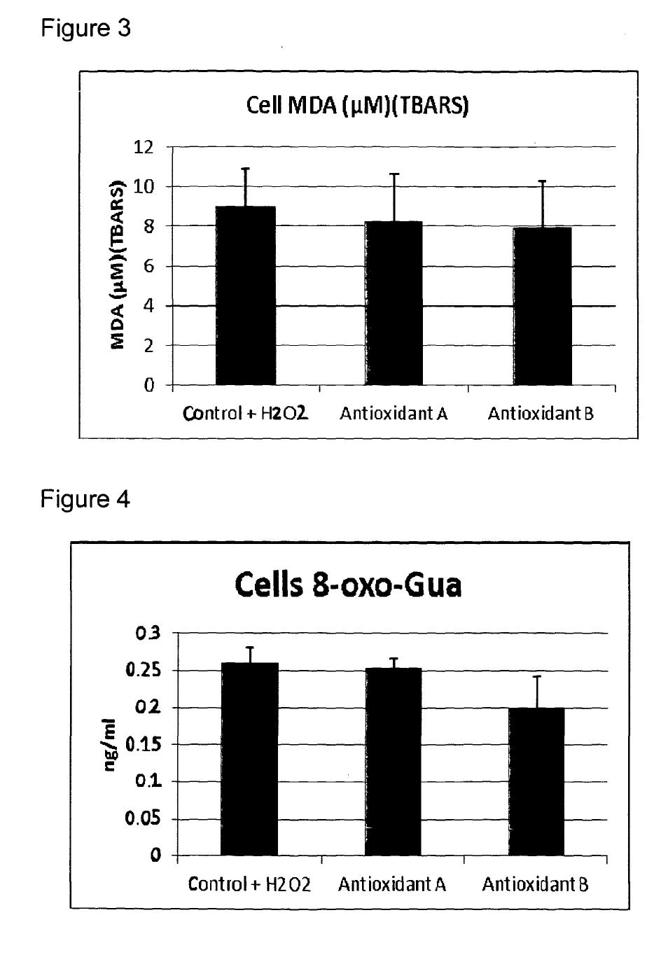

FIG. 3 shows the level of lipid peroxidation in the cell fraction measured by the marker MDA. The values are the average from two separate cell isolations.

FIG. 4 shows the level of DNA damage in the cell fraction measured by the marker 8-oxo-Gua. The values are the average from two separate cell isolations.

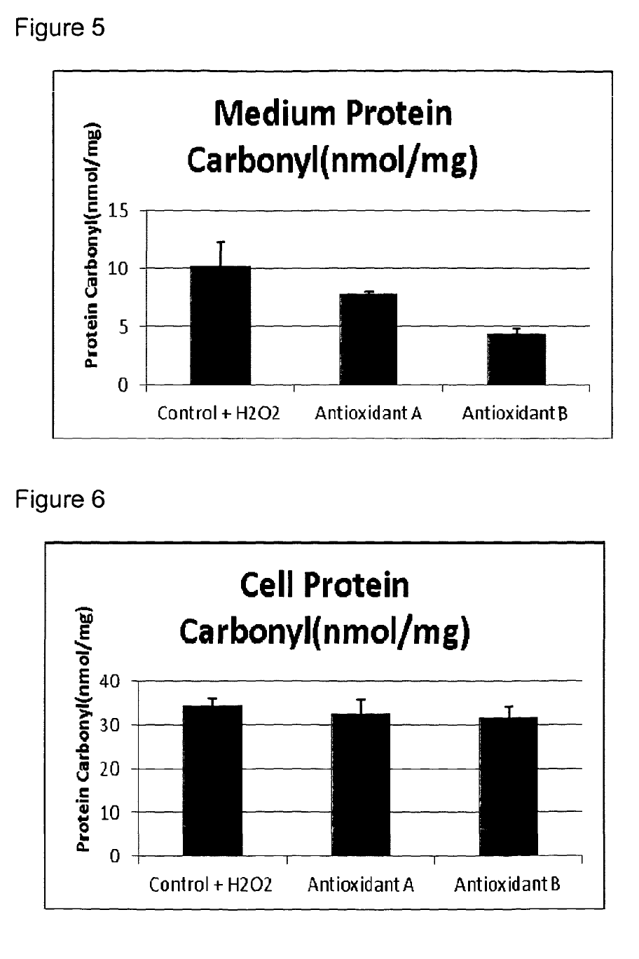

FIG. 5 shows the level of oxidized protein in the medium fraction measured by the marker protein carbonyl. The values are the average from two separate cell isolations.

FIG. 6 shows the level of oxidized protein in the cell fraction measured by the arker protein carbonyl. The values are the average from two separate cell isolations.

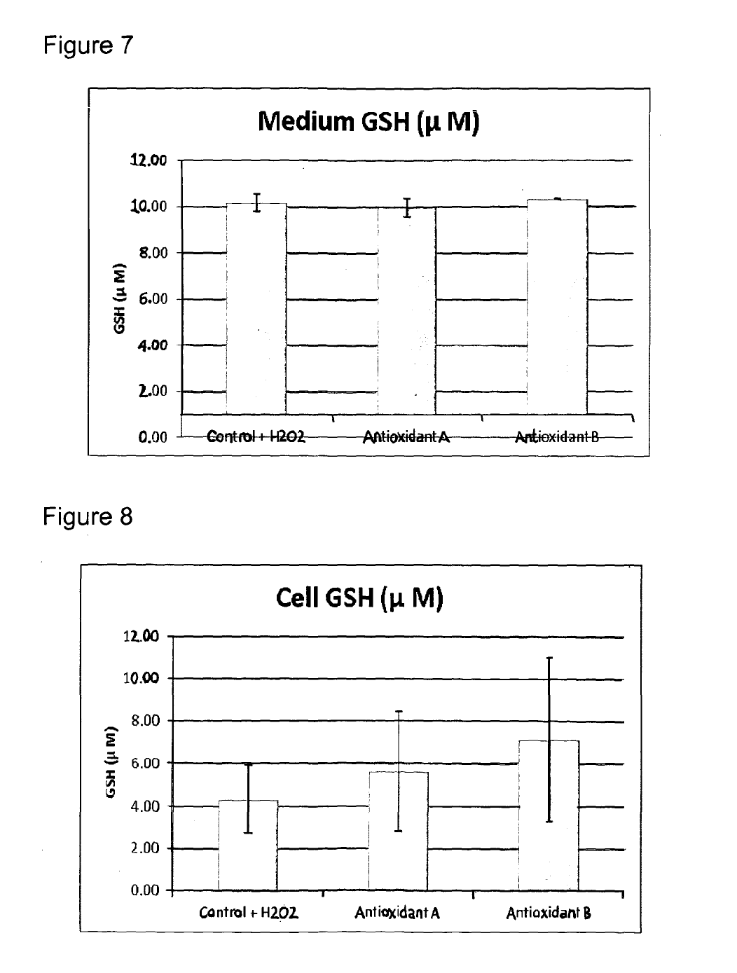

FIG. 7 shows the level of antioxidant capacity in the medium fraction measured by the level of the endogen antioxidant GSH. The values are the average from two separate cell isolations

FIG. 8 shows the level of antioxidant capacity in the cell fraction measured by the level of the endogen antioxidant GSH. The values are the average from two separate cell isolations

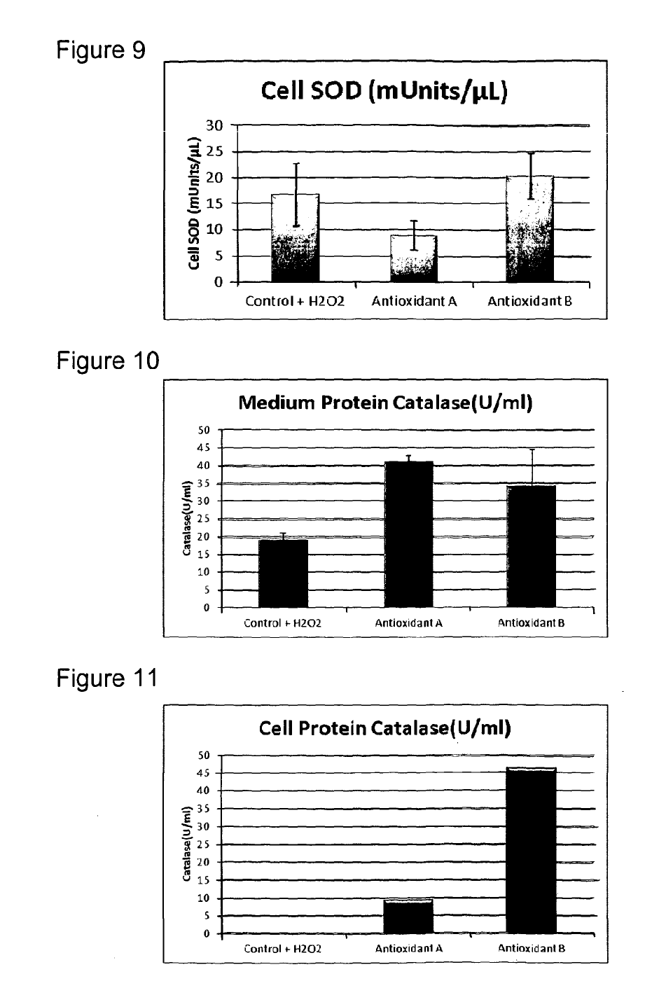

FIG. 9 shows the level of antioxidant capacity in the cell fraction measured by the level of the endogen antioxidant SOD. The values are the average from two separate cell isolations

FIG. 10 shows the level of antioxidant capacity in the cell fraction measured by the level of the endogen antioxidant catalase. The values are the average from two separate cell isolations

FIG. 11, shows the level of antioxidant capacity in the cell fraction measured by the level of the endogen antioxidant catalase. The values are the average from two separate cell isolations

### EXAMPLES

### Example 1

### Example Preparation of Compositions

All references to (w/w) % of the formulation components present in the capsules used throughout the Examples are based on the total filling weight of the capsules, i.e. excluding the weight of the capsule shell. All references to (w/w) % of the capsule shell components are based on the total weight of the capsule shell.

The following capsules are described:

### Placebo: Olive oil

### Composition A:

| | | | Conc. | |
|---|---|---|---|---|
| | | Conc. | (% w/w) of | Amount of |
| | Amount | (% w/w) of | component in | component- |
| | of | component | component- | containing |
| | component | in | containing | product per |
| Component | per capsule | formulation | Product | capsule |
| Astaxanthin | 2 mg | 2 | 5 | 40 mg |
| (present as | | | | |
| Bioastin) | | | | |
| Lutein | 6 mg | 6 | 21% (combined | 34.3 mg |
| Zeaxanthin | 1.2 mg | 1.2 | total:Lutein and | |
| | | | Zeaxanthin | |
| | | | provided | |
| | | | in a combined | |
| | | | product in | |
| | | | a 5:1 ratio) | |
| Safflower | 25.7 mg | 25.7 | 100 | 25.7 mg |
| Oil Type (II) | | | | |
| (Excipient) | | | | |
| | | | Total Filling | 100 mg |
| | | | Weight | |

Capsule Shell: Gelatin (fish): 57.20 mg per capsule Glycerin 99.5%: 26.24 mg per capsule Water, pur.: 10.56 mg per capsule
Average capsule weight: 194 mg+/−10%
Average filling weight: 100 mg+/−10%
Average shell weight: 94 mg+/−10%

### Composition A*:

Lutein, 6 mg/Capsule; Astaxanthin, 2 mg/Capsule and Zeaxanthin, 1.2 mg/Capsule; in addition to standard excipients such as lecithin, the capsule shell comprising gelatin 63.41%, glycerine 29.09% and water 7.49%.

### Composition B:

| | | | Conc. | |
|---|---|---|---|---|
| | | Conc. | (% w/w) of | Amount of |
| | Amount | (% w/w) of | component in | component- |
| | of | component | component- | containing |
| | component | in | containing | product per |
| Component | per capsule | formulation | Product | capsule |
| Astaxanthin | 2 mg | 2 | 5 | 40 mg |
| (present as | | | | |
| Bioastin) | | | | |
| Lutein | 6 mg | 6 | 21% (combined | 34.3 mg |
| Zeaxanthin | 1.2 mg | 1.2 | total:Lutein and | |
| | | | Zeaxanthin | |
| | | | provided | |
| | | | in a combined | |
| | | | product in | |
| | | | a 5:1 ratio) | |
| Safflower | 25.7 mg | 25.7 | 100 | 25.7 mg |
| Oil Type (II) | | | | |
| (Excipient) | | | | |
| | | | Total Filling | 100 mg |
| | | | Weight | |

Capsule Shell: Gelatin (fish): 130.00 mg per capsule Glycerin 99.5%: 59.64 mg per capsule Water, pur.: 15.36 mg per capsule
Average capsule weight: 765 mg+/−8.2%
Average filling weight: 560 mg+/−7.5%
Average shell weight: 205 mg+/−10%

### Composition B*:

300 mg of Krill oil, 20.6 mg of Vitamin E, 176.5 mg of Vitamin C, 34.3 mg of lutein/zeaxanthin in a ratio of 5:1, 22 mg of astaxanthin in addition to standard excipients such as lecithin, the capsule shell comprising gelatin 63.41%, glycerine 29.09% and water 7.49%.

### Composition C:

| | | | Conc. | |
|---|---|---|---|---|
| | | Conc. | (% w/w) of | Amount of |
| | Amount | (% w/w) of | component in | component- |
| | of | component | component- | containing |
| | component | in | containing | product per |
| Component | per capsule | formulation | Product | capsule |
| Astaxanthin | 2 mg | 2 | 5 | 40 mg |
| (present as | | | | |
| Bioastin) | | | | |
| Lutein | 6 mg | 6 | 21% (combined | 34.3 mg |
| Zeaxanthin | 1.2 mg | 1.2 | total:Lutein and | |
| | | | Zeaxanthin | |
| | | | provided | |
| | | | in a combined | |
| | | | product in | |
| | | | a 5:1 ratio) | |
| Safflower | 25.7 mg | 25.7 | 100 | 25.7 mg |
| Oil Type (II) | | | | |
| (Excipient) | | | | |
| | | | Total Filling | 100 mg |
| | | | Weight | |

Capsule Shell: Gelatin (275 Bloom F): 152.00 mg per capsule Glycerin 99.5%: 69.73 mg per capsule Water, pur.: 28.27 mg per capsule
Average capsule weight 923 mg+/−8.2%
Average filling weight: 673 mg+/−7.5%
Average shell weight: 250 mg+/−10%

### Example 2

### Total Antioxidant Power

The total antioxidant capacity of different formulations can be compared by measuring the antioxidant activity against some of the most important pro-oxidants causing oxidative damage in the human body. In combination, they cause DNA, protein, and lipid damage and contribute to systemic inflammation and other harmful pathways.

The antioxidant power of formulations A and B from Example 1 was measured by the following test tube analysis:
FRAP—ferric reducing antioxidant power—this is an analysis which measures the ability of a composition to transfer electrons to iron ions, i.e. its ability prevent iron oxidising.
In addition, the antioxidant capacity of a composition was estimated by measuring its effect on reactive oxygen components like peroxyl radicals, peroxynitrite and superoxide anion (NORAC—Peroxynitrite Radical Averting Capacity, HORAC—Hydroxyl Radical Averting Capacity, SOD—Superoxide radical absorbance capacity)

The FRAP analysis was performed at an independent contract laboratory; Brunswick Laboratories, 50 Commerce Way Norton, Mass. 02766, USA. Brunswick Laboratories fully complies with Good Laboratory Practices (GLP, 21 CFR Part 58) guidelines. The results are shown in Table A below:

TABLE A
FRAP analysis 1

| | Composition A | Composition B |
|---|---|---|
| μmol TE/gram | 151 | 510 |

Composition B had a 237.75% higher antioxidant capacity compared to position A measured by FRAP.

The FRAP study was then repeated at a chemical analysis contract lab Vitas AS, Gaustadallëen 21, 0349 Oslo, Norway. The results are shown in Table B below:

TABLE B
FRAP analysis 2 (Performed by Vitas AS)

| | Composition A | Composition B |
|---|---|---|
| μmol/capsule | 6.17 | 182.4 |

Composition B had a 2856.24% higher antioxidant capacity compared to composition A measured by FRAP.

The free radicals analysis was performed by Brunswick laboratories. The results, measured in μmole TE/gram, are shown in Table C, below:

TABLE C
Free Radical Analysis (μmole TE/gram)

| | Composition | Composition |
|---|---|---|
| | A | B |
| Antioxidant power against peroxyl radicals | 71 | 575 |
| Antioxidant power against peroxynitrite | 1 | 34 |
| Antioxidant power against superoxide anion | 178 | 2333 |

The antioxidant capacity of composition B against peroxyl radicals was 710% increased compared to composition A. The antioxidant capacity of composition B against peroxynitrite was 3300% increased compared to composition A. The antioxidant capacity of composition B against superoxide anion was 1211% increased compared to composition A.

Conclusion: Antioxidant composition B had a greater antioxidant power than composition A measured by FRAP, antioxidant power against peroxyl radicals, antioxidant power against peroxynitrite and antioxidant power against superoxide anion.

### Example 3

### Protection of Muscle Cells Against Oxidative Stress

The protective effect of Compositions A and B from Example 1 on oxidative damage in muscle cells was compared.

Oxidative stress and antioxidant power can be difficult to measure because many reactive oxygen species (ROS) have a very short lifetime. It is common practice to measure oxidative damage to bio molecules as a measure of the degree of oxidative stress. Oxidative damage of these bio molecules is known to be involved in the development of a number of diseases.

Some of these damaged markers can accumulate in the body in both abnormal processes and by aging. Frequently used biomarkers are:
Oxidized lipid: Oxidation of lipids leads to the formation of several end products like malondialdehyd (MDA) and isoprostanes. These end products can be measured in blood and urine. F₂-isoprostanes are formed by free radical-catalyzed peroxidation of esterified arachidonic acid before it is slit and released to the circulation. Oxidized prostaglandin can be involved in the mechanism behind atherosclerosis and can act as a mutagen. In addition 8isoPGF2 can be able to modify the fluidity and integrity of membranes. It can be measured in both blood and urine and is considered to be a good marker of oxidative stress.
Oxidized proteins: Protein carbonyls are formed by oxidation of several amino acid chains and by the formation of advanced glycated end products. The blood concentration of these is a marker of oxidative stress.
Oxidative damage of DNA: Oxidation of genetic material (DNA) is particularly associated with the risk of cancer. There are many different oxidized DNA products. A common marker is 8-hydroxy-20-deoxyguanosine (8OHdG). 8OHdG is one of the most common DNA lesions resulting from reactive oxygen species and is used as a sensitive marker for oxidative stress. The lesion can result in a mismatched pairing and result in substitutions in the genome. Repair mechanisms results in excretion of 8-oxo-Gua from the intracellular to extracellular milieu including the blood and urine.

### Rat Neonatal Cardiomyocyte Stimulated by Oxidative Stress.

H₂O₂ treatment is a physiologically relevant and easily manipulatable form of oxidative stress. Normal human H₂O₂ concentration in blood is 20 μmol/L and can reach 40 μmol/l during infections. In rat normal H₂O₂ concentrations are about 3 μmol/L and in diabetic rats the level raises to about 6 umol/L. Concentrations >100 μmol/L promote cardiac myocyte apoptosis. The possible protective effect of pre-treatment of rat neonatal cardiomyocytes with antioxidants against H₂O₂ induced oxidative stress was investigated. Markers of oxidative stress 8-OH-GUA, 8-iso-PGF2a, TBARS, SOD, catalase, protein carbonyl and selected cytokines was evaluated by Vitas, AS Gaustadallëen 21, 0349 Oslo, Norway.

All cells were pretreated for 2 hours with/without composition A or B from Example 1 or olive oil as a control before treatment with 50 μMol/L H₂O₂ in the corresponding media in 6 well cell culture plates. Cells were removed from the media by centrifugation at 1000×g for 10 minutes at 4° C. The attached cells were washed with ice cold PBS and scraped from the wells. Cell nn media fractions were stored in freezer in the presence of protease inhibitors before analyzed.

a) Lipid Peroxidation Measured by 8-iso-PGF2

8-iso-PGF2a was measured in the cell fraction by LC-MS IMS. The values the average from two separate cell isolations.

In the medium fraction composition A had a small but not significant effect reducing lipid peroxidation by 1.9%. Composition B decreased lipid peroxidation measured by 8-iso-PGF2 by 23% as shown in Table 1 below and in FIG. 1:

TABLE 1
8-iso-PGF2a level in the medium fraction

| Medium | 8-iso-PGF2a (ng/ml) | Stdev | % reduction |
|---|---|---|---|
| Control | 0.260 | 0.021 | |
| Composition A | 0.255 | 0.010 | 1.9% |
| Composition B | 0.200 | 0.042 | 23% |

In the cell reaction the standard deviation between the two cell isolations was too large to conclude if composition A had an effect on lipid peroxidation. Composition B decreased lipid peroxidation by 86.8%, as shown in Table 2 below and in FIG. 2:

TABLE 2
8-iso-PGF2a level in the cell fraction

| Cell | 8-iso-PGF2a (ng/ml) | Stdev | % reduction |
|---|---|---|---|
| Control | 2.155 | 0.696 | |
| Composition A | 2.505 | 1.170 | |
| Composition B | 0.285 | 0.116 | 86.8% |

### b) Lipid Peroxidation Measured by MDA

MDA was measured to estimate the effect of the antioxidant compositions on lipid peroxidation. The values are the average from two separate cell isolations.

MDA level in the medium fraction was under the detection limit. In the cell fraction composition A decreased lipid peroxidation by 8.02% while composition B resulted in a 11.69% decrease, as shown in Table 3 below and in FIG. 3:

TABLE 3
MDA level in the cell fraction

| Cell | MDA (μM) | Stdev | % reduction |
|---|---|---|---|
| Control | 8.98 | 1.87 | |
| Composition A | 8.26 | 2.33 | 8.02 |
| Composition B | 7.93 | 2.32 | 11.69 |

c) DNA Oxidation Measured 8-oxo-Gua

8-oxo-Gua was measured in the cell fraction by LC-MS/MS. The values are the average from two separate cell isolations.

In the medium fraction the level of 8-oxo-Gua was under the detection limit in all samples. In the cell fraction composition A reduced the level of 8-oxo-Gua by 1.9% while antioxidant solution B decreased the level of 8-oxo-Gua by 23%, as shown in Table 4 below and in FIG. 4:

TABLE 4
8-oxo-Gua A level in the cell fraction

| Cell | 8-oxo-Gua (ng/ml) | Stdev | % reduction |
|---|---|---|---|
| Control | 0.260 | 0.021 | |
| Composition A | 0.255 | 0.010 | 1.9 |
| Composition B | 0.200 | 0.042 | 23.0 |

### d) Protein Oxidation Measured by Protein Carbonyl

Protein carbonyl was measured in the cell fraction by ELISA. The values are the average from two separate cell isolations.

In the medium fraction composition A had a significant effect reducing the protein carbonyl level by 23.4%, while Composition B had an even greater effect reducing the protein carbonyl level by 56.6%, as shown in Table 5 below and In FIG. 5:

TABLE 5
Protein carbonyl level in the medium fraction

| Medium | Protein Carbonyl (nmol/mg) | Stdev | % reduction |
|---|---|---|---|
| Control | 10.25 | 2.015 | |
| Composition A | 7.85 | 0.176 | 23.4 |
| Composition B | 4.45 | 0.318 | 56.6 |

In the cell fraction composition A reduced the level of protein carbonyl by 5.5%, while Antioxidant solution B decreased the level of protein carbonyl by 8.0%, as shown in Table 6 below and in FIG. 6:

TABLE 6
Protein carbonyl level in the cell fraction

| Medium | Protein Carbonyl (nmol/mg) | Stdev | % reduction |
|---|---|---|---|
| Control | 34.45 | 1.66 | |
| Composition A | 32.55 | 3.36 | 5.5 |
| Composition B | 31.70 | 2.47 | 8.0 |

### Antioxidant Levels

The level of antioxidants in the body is an indication of the amount of oxidative stress. Glutathione in the blood is thought to provide a measure of the level in other parts of your body and lowering the concentration of both reduced glutathione (GSH) and glutathione disulfide (GSSG) is used as an indicator of oxidative stress. Also, other antioxidants such as SOD, GPX, and catalase and glutathione reductase can be measured.

### e) Antioxidant Capacity Measured by GSH

Total GSH was measured in the cell fraction by HPLC-FLD. The values are the average from two separate cell isolations.

The GSH level in the medium fraction was not significant affected by the antioxidant compositions but composition B resulted in a slight 1.6% increase in GSH level, as shown in Table 7 below and in FIG. 7:

TABLE 7
GSH level in the medium fraction

| Medium | GSH (μM) | Stdev | % increase |
|---|---|---|---|
| Control | 10.17 | 0.39 | |
| Composition A | 9.98 | 0.38 | |
| Composition B | 10.33 | 0.01 | 1.6 |

In the cell fraction the standard deviations were too high to conclude there was an effect. But there was a tendency that both composition A and B could increase the GSH antioxidant capacity by 23.3% and 39.4%, as shown in Table 8 below and in FIG. 8:

TABLE 8
GSH level in the cell fraction

| Medium | GSH (μM) | Stdev | % increase |
|---|---|---|---|
| Control | 4.31 | 1.61 | |
| Composition A | 5.61 | 2.81 | 23.3 |
| Composition B | 7.10 | 3.87 | 39.4 |

### f) Antioxidant Capacity Measured by SOD

Total SOD was measured in the cell fraction by ELISA. The values are the average from two separate cell isolations.

In the medium fraction the level of SOD was under the detection limit in all samples. In the cell fraction composition B increased the GSH antioxidant capacity by 18%, as shown in Table 9 below and in FIG. 9:

TABLE 9
SOD level in the cell fraction

| Medium | SOD (mUnits/μL) | Stdev | % increase |
|---|---|---|---|
| Control | 16.61 | 6.059 | |
| Composition A | 8.76 | 2.768 | |
| Composition B | 20.25 | 4.401 | 18.0 |

### g) Antioxidant Capacity Measured by Protein Catalase

Protein catalase was measured in the cell fraction by ELISA. The values are the average from two separate cell isolations.

The protein catalase level was under the detection limit in most of the cell samples. The antioxidant capacity measured by protein catalase level in the medium fraction was increased by both antioxidant compositions. Composition A resulted in a 53.5% increase while composition B increased the level by 44.1%, as shown in Table 10 below and in FIG. 10:

TABLE 10
Protein Catalase level in the medium fraction

| Medium | Protein Catalase(U/ml) | Stdev | % increase |
|---|---|---|---|
| Control | 19.15 | 1.750 | |
| Composition A | 41.16 | 1.527 | 53.5 |
| Composition B | 34.26 | 10.18 | 44.1 |

In samples from one of the cell isolations, the protein catalase level was measurable after antioxidant treatment. Composition B increased the level significantly more than composition A, as shown in Table 11 below and in FIG. 11:

TABLE 11
Protein Catalase level in the cell fraction

| | | | |
|---|---|---|---|
| Cell | Protein Catalase (U/ml) | Stdev | % increase |

| | |
|---|---|
| Control | <1.56 |
| Composition A | 9.24 |
| Composition B | 46.38 |

Conclusion: Antioxidant composition B had a greater effect than composition A on lipid peroxidation measured by the specific marker 8-iso-PGF2a and MDA and oxidative damage on protein and DNA measured by protein carbonyl and 8-oxo-Gua. In addition composition B was more effective than composition A in increased the antioxidant capacity measured by GSH and SOD.

### Example 4

### Luminex® Extracellular Assay

As evidence for the activity of a composition according to the present invention, the Luminex® Extracellular Assay, commercially available from the company Invitrogen was used. In that setting, two compositions were compared with each other and with a placebo. The first of the two compositions (Composition A*) is the commercially available product VitaePro® and the second composition (Composition B*) is the composition including the formulation according to the present invention. The Assay used is based on evaluating markers which are descriptive for muscle activity and inflammation in the body.

### Wash Solution and Standard Preparation:

A Wash Solution was prepared according to the Luminex® Extracellular Assay Protocol by diluting the entire contents of the 20× Wash Solution bottle with 285 ml ddH2O. The Assay Standard was prepared by reconstituting the lyophilized standard in 100% Assay Diluent (serum and plasma samples) or 50% Assay Diluent/50% tissue culture media (tissue culture supernatants). The preparation was rehydrated at room temperature for 8-10 minutes and the vials were gently inverted several times and let sit an additional 3-5 minutes to ensure complete hydration. 3-fold serial dilutions of the reconstituted standard were performed to prepare a seven point standard curve.

### The Further Steps:

A. Analyte Capture:

1, Vortex (30 sec) and sonicate (30 sec) the 10× Capture Bead stock. In a foil wrapped tube, dilute the 10× Capture Bead stock (2.5 μl per well) in Working Wash Solution (25 μl per well). For higher multiplexing adjust the volume of Working Wash Solution to account for the extra volumes of 10× Capture Bead stocks retained. (Refer to Calculation Worksheet).
2. Pre-wet the standard and sample wells h 200 μl Working Wash Solution. Remove liquid from the plate with a vacuum manifold.
3. Vortex (30 sec) and sonicate (30 sec) the diluted Capture Bead solution. Immediately add 25 μl to each assay well followed by 200 μL of 1× Wash Solution. Aspirate and repeat the wash with 200 μL of Working Wash Solution. Tap and dab the bottom of the filter plate as needed.
4. Add 50 μl Incubation Buffer to all assay wells.
5. Add 100 μl standard into designated wells and cover and incubate the plate or 2 hours at room temperature on an orbital plate shaker (500-600 rpm).

B. Analyte Detection

6. Prepare 1× Biotinylated Detector Antibody: Dilute the 10× Biotinylated Detector Antibody (10 μl per well) in Biotin Diluent (100 μl per well). Aspirate and wash the assay wells twice with 200 μl Working Wash Solution. Add 100 μl diluted Biotinylated Detector Antibody to each assay well. Cover and incubate the plate for 1 hour on a plate shaker (500-600 rpm).
7. Prepare 1× Streptavidin-RPE solution: Dilute the 10× Streptavidin-RPE (10 μl per well) in RPE-Diluent (100 μl per well) in a foil wrapped tube. Aspirate and wash the assay wells twice with 200 μl Working Wash Solution. Add 100 μl diluted Streptavidin-RPE to each assay well. Cover and incubate the plate for 30 minutes on a plate shaker (500-600 rpm).

C. Assay Reading

8. Aspirate and wash the assay wells 3 times with 200 μl Wash Solution. Add 100 μl Wash Solution to each assay well and place the plate on the plate shaker (500-600 rpm) for 2-3 minutes.
9. Read the plate on a Luminex® instrument.

Results: The levels of the following markers are assessed: Adiponectin, Aggregated Aβ, Aggregated α-Synuclein, Aβ40, Aβ42, BDNF, DR5, EGF, Eotaxin/CCL11, FGF basic, G-CSF, GDNF, GM-CSF, GRO-α, HGF, IFN-α, IFN-γ, IL-1α, IL-1β, IL-1RA, IL-2, IL-2R, IL-3, IL-4, IL-5, IL-6, IL-6 US, IL-6R, IL-7, IL-8, IL-9, IL-10 hu 9/rt, IL-12 (p40/p70), IL-12 (p70) hu 42/ms, IL-13, IL-15, IL-16, IL-17, Insulin, IP-10, KC, Leptin, MCP-1/CCL2, MCP-2/CCL8, MCP-3/CCL7, MIG/CXCL9, MIP-1α/CCL3, MIP-1β/CCL4, MIP-3β, PDGF-BB, RANTES/CCL5, Resistin, Serum Amyloid A (SAA), Tau (total), Tau [pT181], Tau [pS199], TGF-β1 (activated/treated), TNF-RI, TNF-RII, TNF-α, VEGF.

### Example 5

### Total Antioxidant Power

The total antioxidant capacity of different formulations can be compared by measuring the antioxidant activity against some of the most important pro-oxidants causing oxidative damage in the human body. In combination, they cause DNA, protein, and lipid damage and contribute to systemic inflammation and other harmful pathways.

### Composition A*:

Lutein, 6 mg/Capsule; Astaxanthin, 2 mg/Capsule and Zeaxanthin, 1.2 mg/Capsule; in addition to standard excipients such as lecithin, the capsule shell comprising gelatin 63.41%, glycerine 29.09% and water 7.49%

### Composition B*:

300 mg of Krill oil, 20.6 mg of Vitamin E, 176.5 mg of Vitamin C, 34.3 mg of lutein/zeaxanthin in a ratio of 5:1, 22 mg of astaxanthin in addition to standard excipients such as lecithin, the capsule shell comprising gelatin 63.41%, glycerine 29.09% and water 7.49%.

### Placebo: Olive oil

The antioxidant power of the different formulations will be measured by the following test tube analysis:
FRAP—ferric reducing antioxidant power
ORAC hydro—Oxygen Radical Absorbance Capacity
ORAC lipo
ORAC total
SOAC—singlet oxygen absorbance capacity
NORAC—Peroxynitrite Radical Averting Capacity
HORAC—Hydroxyl Radical Averting Capacity
SOD—Superoxide radical absorbance capacity

The analysis will be performed at an independent contract laboratory; Brunswick Laboratories, 50 Commerce Way Norton, Mass. 02766, USA. Brunswick Laboratories fully complies with Good Laboratory Practices (GLP, 21 CFR Part 58) guidelines.

### Example 6

### Differential Protection Against Oxidative Stress

The protective effect of the formulations from Example 5 on oxidative damage in muscle cell; will be compared.

Lipid peroxidation, as malondialdehyde (DA), will be measured with thiobarbituric acid reaction.

Muscle cells, like for instance L6 myoblasts cells, will be grown and pre-incubated with the composition A or B or placebo before incubation with pro-oxidants. After harvesting, cells will be suspended in PBS and sonicated. The sonicated material will be added to butylated hydroxytoluene. Fifteen percent trichloroacetic acid, HCl, butylated hydroxytoluene, thiobarbituric acid, and SDS will be added to the sample and vortexed. The color will develop at 95° C. and the reaction will be stopped by cooling on ice.

The samples will be centrifuged and the supernatant from each tube will be transferred to a 96-well plate. The absorbance at 540 nm will be measured with reference to a reagent blank.

Thiobarbituric acid reactive substance (TBAR) will be calculated from a standard curve made with various concentrations of 1,1,3,3-tetraethoxypropane and normalized to the protein concentration of each sample.

Also, oxidative stress damage to musclecells will be determined by measuring markers like F2-isoprostane.

8-hydroxy deoxyguanosine (8-OHdG) and comet assay will be determined as a measure of DNA oxidative damage.

In addition nuclear extracts will be prepared from muscle cells and to measure oxidative stress by determining the translocation of p65 NFkappaB into the cell nucleus and the expression of chemokines regulated by this factor like MCP-1 and CINC-1. The cells natural antioxidant defense will be investigated by measuring cell produced antioxidants like catalase, glutathion and superoxide dismutase.

Oxidative stress is close connected to inflammatory reactions. Inflammatory markers like TNF-alpha, IFN-gamma, IL-1, IL-6, IL-8, IL-12 and 1-15, IL-17 will be used to measure the inflammatory response.

### Example 7

### Use of the Product as a Cardioprotective Agent Against Ischemia-Reperfusion Injury

During myocardial ischemia-reperfusion insults, oxygen radical formation is accelerated and plays a critical role in mediating cellular damage and dysfunction.

In the present study, we are proposing to assess the cardio protective efficacy of oral administration of composition A*:

Lutein, 6 mg/Capsule; Astaxanthin, 2 mg/Capsule and Zeaxanthin, 1.2 mg/Capsule; in addition to standard excipients such as lecithin, the capsule shell comprising gelatin 63.41%, glycerine 29.09% and water 7.49%.

in male Wistar rats over a period of 15 consecutive days in an isolated rat heart model. The effect of composition A* will be compared with a mixture of the two antioxidants vitamin C and E.

After 15 days of oral treatment with composition A* or vitamin C and E, the hearts of ats will be subjected to 30 min of global ischemia followed by 2 h of reperfusion and will be measured for infarct size, apoptosis and cardiac functions. In addition the gene expression profile will be determined.

1.-24. (canceled)

25. A dietary formulation, comprising:
a. an oil obtained from a marine invertebrate;
b. one or more carotenoids; and
c. Vitamin C and/or Vitamin E.

26. The formulation of claim 25, wherein the formulation comprises Vitamin C and Vitamin E.

27. The formulation of claim 25, wherein the one or more carotenoids comprise lutein, zeaxanthin, and astaxanthin.

28. The formulation of claim 25, wherein the formulation comprises:
a. an oil obtained from a marine invertebrate;
b. lutein, zeaxanthin, and astaxanthin; and
c. Vitamin C and Vitamin E.

29. The formulation of claim 25, wherein the oil is obtained from Crustacea or Mollusca.

30. The formulation of claim 29, wherein the oil is obtained from krill or calamari.

31. The formulation of claim 25, wherein the oil obtained from a marine invertebrate is at a concentration of 30% to 90% (w/w), and wherein the one or more carotenoids are at a combined concentration of 0.1 to 10% (w/w).

32. The formulation of claim 31, wherein the oil obtained from a marine invertebrate is at a concentration of 40% to 60% (w/w).

33. The formulation of claim 31, wherein the one or more carotenoids are at a combined concentration of 0.18 to 4.5% (w/w).

34. The formulation of claim 30, wherein the formulation comprises:
a. 225 to 350 mg krill oil or calamari oil;
b. 5 to 7.5 mg lutein, 1 to 1.5 mg zeaxanthin, and 1 to 5 mg astaxanthin; and
c. 10 to 150 mg Vitamin C and 5 to 30 mg Vitamin E.

35. The formulation of claim 30, wherein the formulation comprises:
a. 40 to 60 w/w % krill oil or 35% to 50% calamari oil;
b. 0.75 to 1.35 w/w % lutein, 0.15 to 0.28 w/w % zeaxanthin, and 0.25 to 0.4 w/w % astaxanthin; and
c. 2 to 25 w/w % Vitamin C and 1 to 5 w/w % Vitamin E.

36. A composition, comprising: a dietary formulation, wherein the dietary formulation comprises an oil obtained from a marine invertebrate, one or more carotenoids, and Vitamin C and/or Vitamin E; wherein the composition optionally comprises a physiological excipient; and wherein the composition is used in animal feed, a dietary supplement, or a human food product.

37. The composition of claim 36, wherein the composition is in the form of a tablet or formulated in a capsule.

38. A method of combating an excessive exercise-induced or physical activity-induced condition in a subject, comprising: administering to a subject the dietary formulation of claim 25.

39. A method of reducing oxidative muscle stress or muscle damage in a subject, comprising: administering to a subject the dietary formulation of claim 25.

40. The method of claim 38, wherein the excessive exercise-induced or physical activity-induced condition is selected from the group consisting of Delayed Onset Muscle Soreness (DOMS), muscle cramps or spasms, muscle aches, muscle atrophy, muscle injuries like strains and the disruption of muscle fibers, muscle weakness and heaviness, metabolic muscle disorders, muscle soreness, muscle fatigue, muscle stiffness, and lack of muscle flexibility.

41. The method of claim 39, wherein the oxidative muscle stress or muscle damage is the result of Delayed Onset Muscle Soreness (DOMS), muscle cramps or spasms, muscle aches, muscle atrophy, muscle injuries like strains and the disruption of muscle fibers, muscle weakness and heaviness, metabolic muscle disorders, muscle soreness, muscle fatigue, muscle stiffness, and/or lack of muscle flexibility.
